(19) United States
(12) Patent Application Publication
(10) Pub. No.: US 2020/0000435 A1
(43) Pub. Date: Jan. 2, 2020

(54) ULTRASOUND APPARATUS AND METHOD

(71) Applicant: CARESTREAM HEALTH, INC., Rochester, NY (US)

(72) Inventor: Ajay ANAND, Rochester, NY (US)

(21) Appl. No.: 16/022,886

(22) Filed: Jun. 29, 2018

(51) Int. Cl. A61B 8/08; A61B 8/00

(52) U.S. Cl. CPC A61B 8/5207; A61B 8/46; A61B 8/565; A61B 8/587

(57) ABSTRACT

A method for ultrasound imaging generates and emits a test pattern of ultrasound transmission signals from a transducer probe. The generated test pattern is sensed and one or more defective transducer elements of the transducer probe identified. Data is recorded related to the one or more defective transducer elements and the recorded data is associated with the transducer probe. At least one ultrasound image is acquired and displayed using the transducer probe and using the associated recorded data for the transducer probe.

### TECHNICAL FIELD

The disclosure relates generally to the field of medical diagnostic ultrasound systems and methods and more particularly to apparatus and methods that provide improved tracking of ultrasound system performance.

### BACKGROUND

Ultrasound imaging systems/methods are well known. See for example U.S. Pat. No. 6,705,995 (Poland) and U.S. Pat. No. 5,370,120 (Oppelt), incorporated herein by reference in their entirety. Ultrasound imaging uses a transducer that transmits a high-frequency sound signal into the tissue or other sample that is to be examined and forms an image according to modulation imparted to the reflected signal from the sample.

Proper functioning of the ultrasound transducer is a key factor for reliable ultrasound diagnosis. This function depends on the condition of the numerous piezoelectric elements in the transducer array used to generate and sense the ultrasound signals, on the integrity of the acoustic coupling maintained between the transducer elements and the transducer interface surface, and on wiring that provides the needed signal connections with each of the transducer elements.

Transducer performance can degrade with use, component aging, and handling. Over time, for example, individual transducer array elements can fail to operate consistently or cable interconnections can exhibit intermittent problems. Delamination can occur, in which the transducer elements are separated from impedance matching features. Problems such as these can have undesirable impact on diagnosis and treatment. For example, in a landmark study in 2006 reported by a hospital in Sweden, it was discovered during a re-examination of a patient that a congenital heart disease had gone undetected at an initial examination in 2004. After a check of test results, it was determined that a defective transducer was at fault and should have been replaced.

Only relatively recently have approaches and utilities been developed for systematic transducer testing and quality assurance protocols that help to track transducer performance and flag components that may be in need of repair or replacement. For example, dedicated automated transducer testing devices such as the ProbeHunter™ test system (BBS Medical AB, Sweden) and the Sonora FirstCall test system (Sonora Medical Systems Inc.) have been introduced for testing and reporting on essential transducer parameters according to FDA regulations.

The automated tools described above test the transducer on separate tests equipment, exercising transducer operation separately from the ultrasound system to detect transducer failure or levels of transducer performance that fail to meet operating standards. The hospital or other clinical facility can then repair or replace the failed transducer as needed.

However, there can often be different levels of degraded transducer performance, including conditions where acceptable performance can still be achieved by a transducer that has one or more defective elements in a transducer array. It can be expensive and unnecessary to repair a transducer wherein only a small number of transducers within a larger array exhibit failure. Moreover, if transducer performance is satisfactory even with a few defects, there is no transfer of defect information to the corresponding ultrasound system to allow compensation for this deficiency. In summary, any evidence of transducer degradation is either found by visual image observation or else by subjecting a transducer to testing on one of the specialized transducer test tools such as the ones described above.

Thus, it can be seen that there would be advantages to approaches for ultrasound transducer testing using the ultrasound apparatus instead of separate test equipment and for reporting results that allows performance data to be accessible to the ultrasound system.

### SUMMARY

An object of the present disclosure is to advance the art of ultrasound imaging and to improve overall system performance and efficiency.

These objects are given only by way of illustrative example, and such objects may be exemplary of one or more embodiments of the invention. Other desirable objectives and advantages inherently achieved may occur or become apparent to those skilled in the art. The invention is defined by the appended claims.

According to one aspect of the disclosure, there is provided a method for ultrasound imaging comprising: generating and emitting a test pattern of ultrasound transmission signals from a transducer probe; sensing the generated test pattern and identifying one or more defective transducer elements of the transducer probe; recording data related to the one or more defective transducer elements and associating the recorded data with the transducer probe; and acquiring and displaying or storing at least one ultrasound image using the transducer probe and using the associated recorded data for the transducer probe.

### BRIEF DESCRIPTION OF THE DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIGS. 1A and 1B show exemplary ultrasound systems.

FIG. 2 shows a schematic of an exemplary ultrasound system.

FIG. 3 illustrates a Sonographer using an exemplary ultrasound system.

FIG. 4 shows a displayed ultrasound image.

FIG. 5 is a schematic diagram that shows functional components of a transducer probe according to an embodiment of the present disclosure.

FIG. 6 is a logic flow diagram that shows steps in a test sequence for transducer performance according to an embodiment of the present disclosure.

FIG. 7 is a schematic that shows one exemplary test sequence that can be applied for a linear array.

FIG. 8 is a schematic diagram that shows system response to faulted transponder elements according to an embodiment of the present disclosure.

### DETAILED DESCRIPTION OF THE EMBODIMENTS

The following is a detailed description of the preferred embodiments, reference being made to the drawings in which the same reference numerals identify the same elements of structure in each of the several figures.

As used herein, the term “energizable” relates to a device or set of components that perform an indicated function upon receiving power and, optionally, upon receiving an enabling signal.

In the context of the present disclosure, the phrase “in signal communication” indicates that two or more devices and/or components are capable of communicating with each other via signals that travel over some type of signal path. Signal communication may be wired or wireless. The signals may be communication, power, data, or energy signals. The signal paths may include physical, electrical, magnetic, electromagnetic, optical, wired, and/or wireless connections between the first device and/or component and second device and/or component. The signal paths may also include additional devices and/or components between the first device and/or component and second device and/or component.

In the context of the present disclosure, the term “subject” or “body” or “anatomy” is used to describe a portion of the patient that is undergoing ultrasound imaging. The terms “sonographer”, “technician”, “viewer”, “operator”, and “practitioner” are used to broadly indicate the person who actively operates the sonography equipment.

Medical ultrasound (also known as diagnostic sonography or ultrasonography) is a diagnostic imaging technique based on the application of ultrasound, used to display internal body structures such as tendons, muscles, joints, vessels and internal organs.

Reference is hereby made to US 2015/0141821 by Yoshikawa et al. entitled “Ultrasonic Diagnostic Apparatus and Elastic Evaluation Method” incorporated herein in its entirety by reference.

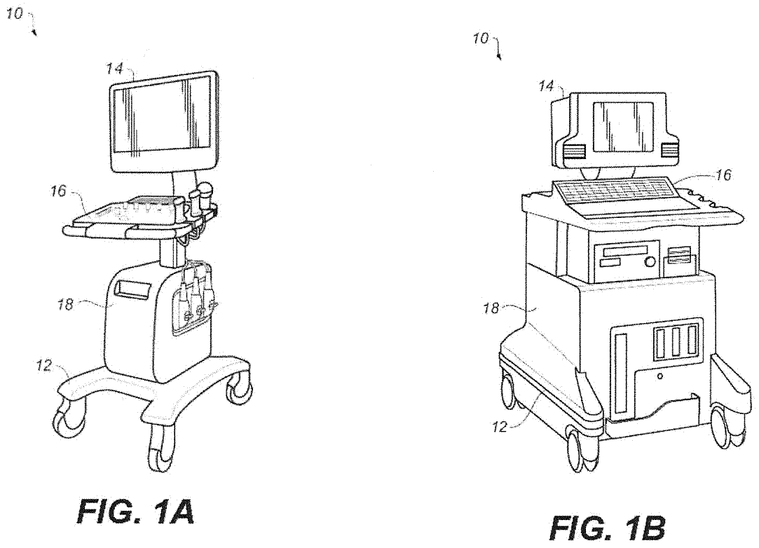

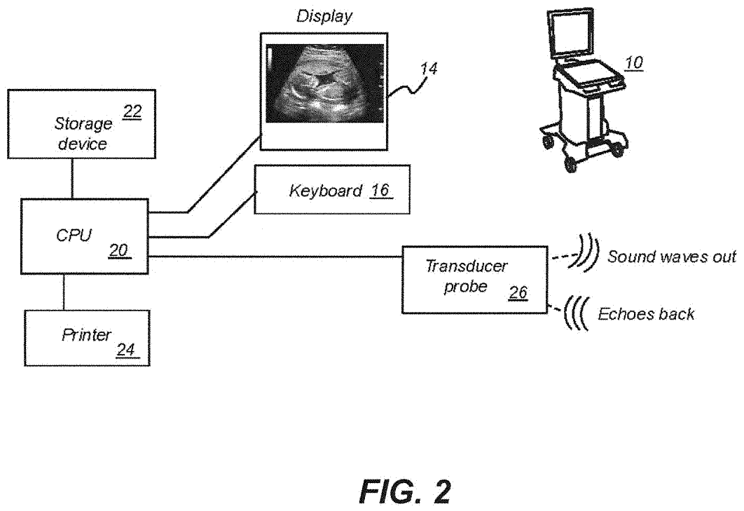

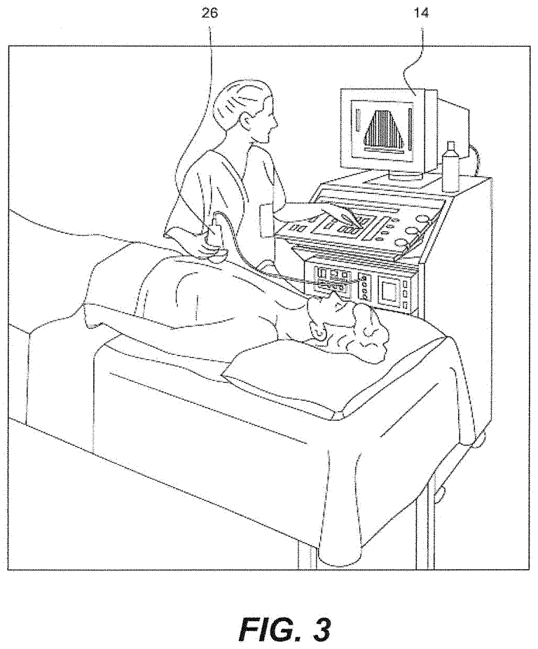

FIGS. 1A, 1B, 2, and 3 show exemplary ultrasound systems 10 including a cart 12 or other mobile base/support, a display monitor 14, an input device (such as keyboard 16 or mouse), and a generator 18. The display 14 can also be a touchscreen to function as an input device for entry of commands to a central processing unit CPU 20 that controls ultrasound system operation. As illustrated, the ultrasound system is a mobile system having wheels. As illustrated in FIGS. 1A and 1B, the ultrasound system 10 can be a mobile or portable system designed to be wheeled from one location to another. As the schematic diagram of FIG. 2 shows, the ultrasound system 10 has a central processing unit CPU 20 that provides control signals and processing capabilities. CPU 20 is in signal communication with display 14 and keyboard or other user interface device 16, as well as with a storage device 22 and an optional printer 24. A transducer probe 26 provides the ultrasound acoustic signal and generates an electronic feedback signal indicative of tissue characteristics according to the echoed sound. FIG. 3 shows an example of an ultrasound exam in progress with an ultrasound image displayed on display monitor 14.

Ultrasound is sound wave energy with frequencies higher than those audible to the human ear. Ultrasonic images, also known as sonograms, are made by directing pulses of ultrasound into tissue using a probe. The sound echoes off the tissue; with different tissues reflecting varying degrees of sound. These echoes are recorded and displayed as an image to the operator.

Different types of images can be formed using sonographic instruments. The most well-known type is a B-mode image, which displays the acoustic impedance of a two-dimensional cross-section of tissue. Other types of images can display blood flow, motion of tissue over time, the location of blood, the presence of specific materials, the stiffness of tissue, or the anatomy of a three-dimensional region.

Typically, the system of FIGS. 1A-3 can be configured to operate within at least two different ultrasound modes. As such, the system provides means to switch between the at least two different ultrasound modes. Such a two-mode configuration and means for switching between modes are well known within the ultrasound technology.

Clinical modes of ultrasound used in medical imaging include the following:
A-mode: A-mode (amplitude mode) is the simplest type of ultrasound. A single transducer scans a line through the body with the echoes plotted on screen as a function of depth. Therapeutic ultrasound aimed at a specific tumor or calculus also uses A-mode emission to allow for pinpoint accurate focus of the destructive wave energy.
B-mode or 2D mode: In B-mode (brightness mode) ultrasound, a linear array of transducers simultaneously scans a plane through the body that can be viewed as a two-dimensional image on screen. Sometimes referred to as 2D mode, this mode is effective for showing positional and dimensional characteristics of internal structures and is generally the starting point for exam types that use other modes.
C-mode: A C-mode image is formed in a plane normal to a B-mode image. A gate that selects data from a specific depth from an A-mode line is used. The transducer is moved in the 2D plane to sample the entire region at this fixed depth. When the transducer traverses the area in a spiral, an area of 100 cm² can be scanned in around 10 seconds.
M-mode: In M-mode (motion mode) ultrasound, pulses are emitted in quick succession. With each pulse, either an A-mode or B-mode image is acquired. Over time, M-mode imaging is analogous to recording a video in ultrasound. As the organ boundaries that produce reflections move relative to the probe, this mode can be used to determine the velocity of specific organ structures.
Doppler mode: This mode makes use of the Doppler effect in measuring and visualizing blood flow.
Color Doppler: Velocity information is presented as a color-coded overlay on top of a B-mode image. This mode is sometimes referred to as Color Flow or color mode.
Continuous Doppler: Doppler information is sampled along a line through the body, and all velocities detected at each point in time are presented (on a time line).
Pulsed wave (PW) Doppler: Doppler information is sampled from only a small sample volume (defined in 2D image), and presented on a timeline.
Duplex: a common name for the simultaneous presentation of 2D and (usually) PW Doppler information. (Using modern ultrasound machines, color Doppler is almost always also used; hence the alternative name Triplex).
Pulse inversion mode: In this mode, two successive pulses with opposite sign are emitted and then subtracted from each other. This implies that any linearly responding constituent will disappear while gases with non-linear compressibility stand out. Pulse inversion may also be used in a similar manner as in Harmonic mode.
Harmonic mode: In this mode a deep penetrating fundamental frequency is emitted into the body and a harmonic overtone is detected. With this method, noise and artifacts due to reverberation and aberration are greatly reduced. Some also believe that penetration depth can be gained with improved lateral resolution; however, this is not well documented.

A sonographer, ultrasonographer, clinician, practitioner, or other clinical user, is a healthcare professional (often a radiographer but may be any healthcare professional with the appropriate training) who specializes in the use of ultrasonic imaging devices to produce diagnostic images, scans, videos, or 3D volumes of anatomy and diagnostic data.

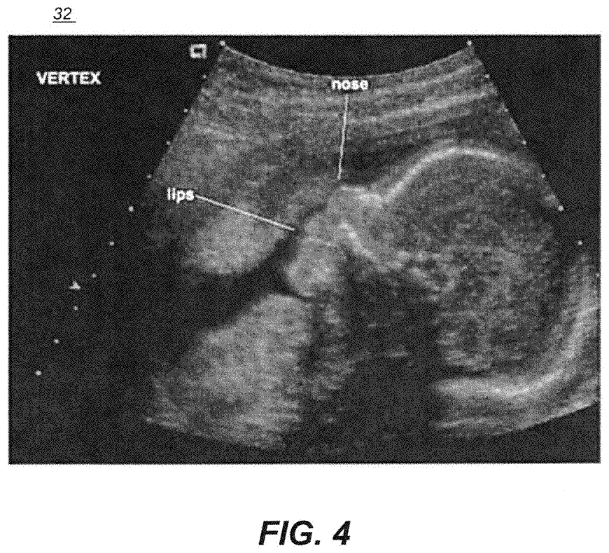

FIG. 4 shows a displayed ultrasound image 32 in grey scale. Such an image can be acquired, for example, using B-mode.

Embodiments of the present disclosure address problems of transducer probe performance testing, tracking, and compensation for various types of transducers that can be used with the ultrasound system. The description that follows describes an embodiment using a linear transducer probe as one exemplary probe type. It should be noted, however, that a similar approach can be used with a transducer probe having a convex shape, a phased-array probe shape, or other type of transducer probe shape, wherein the transducer employs one or more transducer elements.

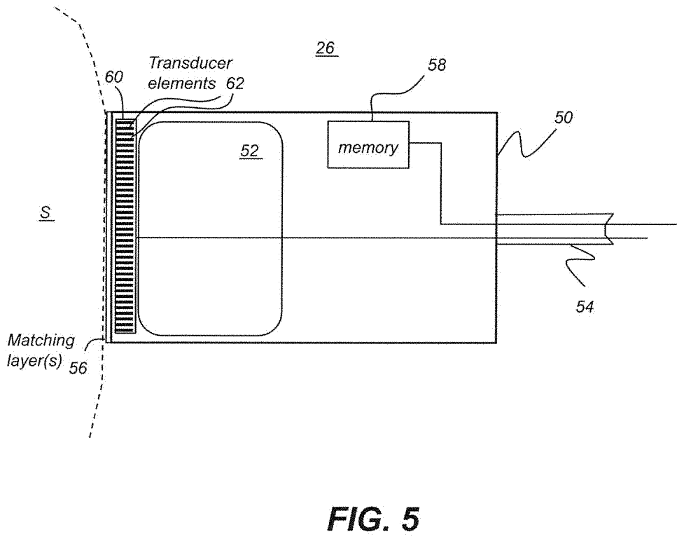

FIG. 5 is a schematic diagram that shows functional components of transducer probe 26 according to an embodiment of the present disclosure. A housing 50-provides the external shell for handling and positioning probe 26 against a subject S. A coaxial cable 54 conveys signals to and from transducer elements 62 in a transducer array 60 and to and from a memory 58. A backing 52 can be provided along with material for acoustic absorption. One or more matching layers 56 provide an acoustic impedance match between the surface of subject S and transducer elements 62.

According to an embodiment of the present disclosure, memory 58 stores a digital “signature” or “personality” that can identify the transducer 26 and can characterize the performance of transducer array 60 elements 62. For example, memory 58, such as an EEPROM component, can record test data that indicates which, if any, of transducer elements 62 are failed or may be unreliable. Memory 58 can store appropriate excitation sequences for various operating modes based on the performance characterization.

### Transducer Test Sequence

An embodiment of the present disclosure uses the ultrasound system 10 itself to test the transducer probe 26 and to generate a signature or personality that can be stored on the probe 26.

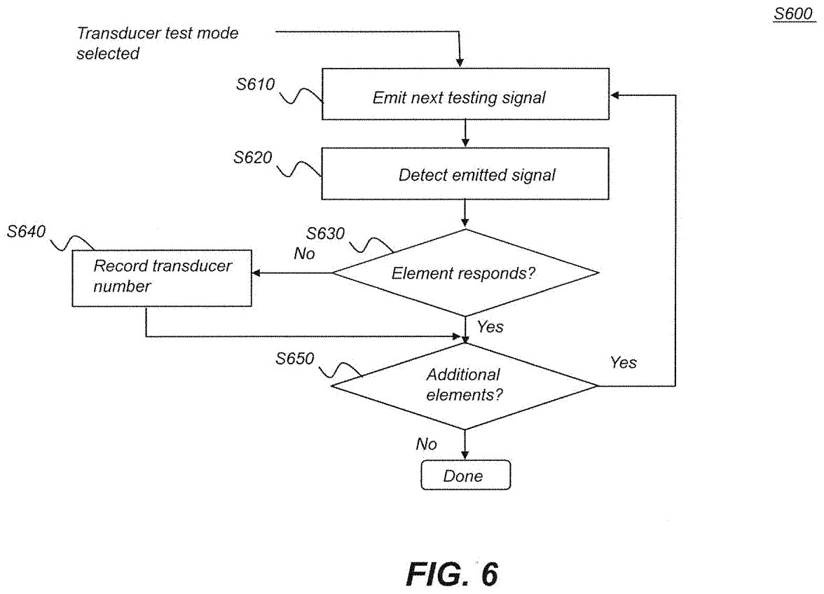

The logic flow diagram of FIG. 6 shows steps in a test sequence S600 for transducer performance that can be executed by the ultrasound system 10. For example, a test mode can be selected by the operator on a control panel or other operator interface. A phantom, such as a container of water having a suitable acoustic reflector can be used for the FIG. 6 test; alternately, the test can be executed without a phantom, such as using ambient air as the sensed “subject”.

The FIG. 6 sequence begins with a signal emission step 610 that emits a testing signal for detection in a detection step S620. The emitted signal can be an acoustic signal that is emitted from a single transducer element or from a pair, bank, or pattern of transducer elements 62 in the array, as described subsequently. Detection step S620 then measures the emitted signal. Step S620 determines whether or not the signal was emitted and whether or not it is detected by transducer 26. A response step S630 determines whether or not transducer element response is normal and, if so, continues to a looping step S650 that repeats the process for a subsequent testing signal in step S610. If transducer element response is faulty, a recording step S640 executes, recording the transducer element number or other identifying data for the failed element in transducer probe memory.

At the end of the FIG. 6 test sequence S600, memory 58 (FIG. 5) is populated with the “signature” of the corresponding transducer probe 26, indicating which elements 62 appear to be fully functional and which elements 62 may be faulty.

The sequence of signals that is emitted can be a straightforward ordinal sequence in which each transducer element is successively actuated and sensed. Alternately, more complex sequences could be used, including actuation patterns that emulate specific imaging modes, for example.

The FIG. 6 test sequence S600 can be executed periodically for each transducer probe, as specified by the biomedical engineering or test department at the hospital, clinic, or other diagnostic facility, or on an as-needed basis.

Test results for the individual transducer can be stored on the transducer probe itself as well as on other networked systems, such as a quality assurance monitoring system with a server that maintains information on equipment status for a facility or department. According to an alternate embodiment, testing results are stored on a central server, a computer that is in signal communication with one or more ultrasound systems and linked to identity information for each probe 26, so that the test results can be downloaded to the ultrasound system when the probe 26 is connected.

According to an alternate embodiment, an online transducer test is provided from an online server by an ultrasound vendor, a test service, or other supplier. For this automated testing, transducer signatures are stored on a central server accessible to the hospital or other ultrasound site. Upon entry of a suitable internet address, and optionally after login with suitable permissions, the vendor or other service identifies the transducer type and executes an automated test sequence, such as the sequence outlined in FIG. 6 or other suitable processing. An appropriate test pattern can be downloaded and verified. Vendor software can then test transducer elements remotely using the downloaded test pattern and characterize transducer performance, including storing the transducer probe signature for future reference. Alternately, an appropriate test program can be downloaded for local testing, optionally with results uploaded for analysis. A suitable transducer excitation scheme can then be downloaded or computed locally for subsequent use.

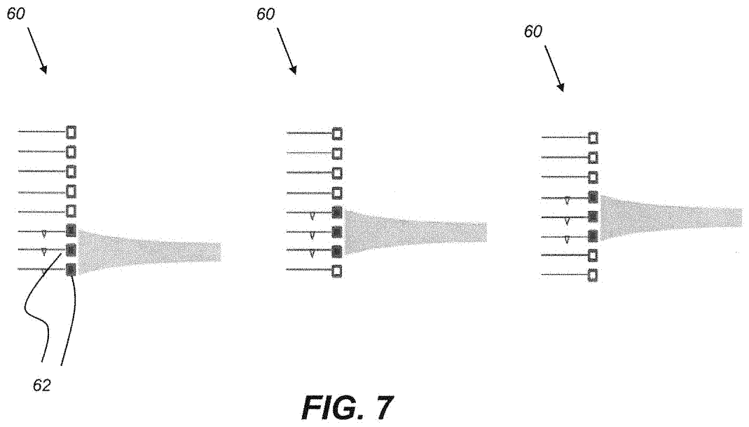

Various types of test sequence can be used. FIG. 7 shows one exemplary test sequence that can be applied for a linear array having 256 elements and used for vascular imaging, such as for evaluation of carotid artery behavior. The beamforming in the portion of linear array 60 shown executes a “marching aperture” scheme as one type of transducer element excitation pattern. Initially, as shown at left in FIG. 7, a few transducer elements 62 at one location along the array 60 are energized, with this pattern repeated along the array 60 to complete testing. Three energized transducer elements 62 are shown; in practice, any number of transducer elements 62 at a time can be energized, or any suitable pattern of transducer elements 62 can be energized for testing in any appropriate sequence. Appropriate delays are applied for transducer energizing and testing. The acoustic beam can be focused and detected along a direction immediately in front of transducer array 60. Successive emissions repeat, using adjoining sets of transducer elements 62 to form acoustic beams for subsequent measurement.

As can readily be observed, in order to image a certain region, the elements directly in front of it are energized. For the case in which a set of adjoining elements fail, it is clear from FIG. 7 that the corresponding region of the subject immediately in front would not be imaged as well. If the failure is significant, voids can appear on the image which, in turn, renders the diagnostic image quality useless. If the failure is less significant, some imaging modes may perform satisfactorily but other modes could fail.

By way of example, B-mode imaging requires less sensitivity compared to color flow and Doppler imaging modes. Hence, while the sonographer may not notice deficiencies in the B-mode image for a system when using a particular transducer probe, the color flow image may be disappointing and may have lost clinical information that is not readily apparent and can lead to an incorrect diagnosis. Hence, having an intelligent system that can not only periodically monitor the status and “health” of the transducer elements but also dynamically adapt the resulting beamforming so that no voids in spatial coverage occur, would be a significant advancement and provide improved ultrasound system operation even when the particular transducer that is deployed has known shortcomings, such as one or more defective or inoperable transducer elements.

### System Response to Transducer Personality Data

Advantageously, an embodiment of the present disclosure stores transducer test results on the transducer probe 26 itself, such as in memory 58 (FIG. 5). When transducer probe 26 is connected to an ultrasound system 10, the system can initially read memory 58 to ascertain the state of the probe 26, such as identifying transducer elements 62 that are faulty, and can configure its corresponding signal patterns appropriately, changing the excitation pattern for the operating transducer elements, for example.

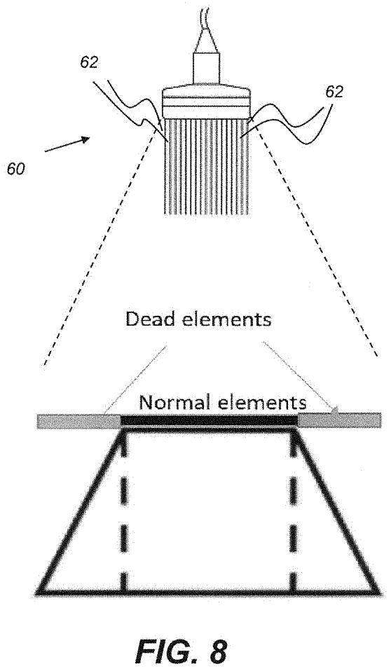

For some operating modes, the ultrasound system 10 can compensate for degraded transducer probe 26 performance by adjusting the pattern of transducer element actuation accordingly. As one example, shown in FIG. 8, the standard imaging format for a linear transducer array 60 is rectangular but end elements 62 of transducer array 60 have become defective or are not functional. As a result, the effective aperture of the array 60 is significantly reduced. Signals generated from array 60 would thus provide reduced spatial coverage.

In the presence of the defect defined above, the imaging format can be immediately switched to a trapezoidal excitation profile, as shown in FIG. 8. This can be achieved by changing the phases applied to the operating transducer elements nearest to the dead transducer elements 62 and having the beam cover the area in front of the dead or defective elements.

In an alternate example, a number of dead transducer elements 62 can be positioned towards the middle of the array 60. In this scenario, once this transducer personality is obtained from memory 58, the system can energize adjacent elements 62 that are functional in order to form the image for the region immediately below the dead elements. This can be achieved by steering the emitted beam rather than by simply energizing transducer elements 62 and measuring signals received at a normal. Compounding techniques can be employed where multiple beams are fired from adjacent elements 62 that lie on both sides of one or more non-functioning elements 62. Such an approach can improve the signal to noise ratio (SNR) for the steered beams. The approach would be optimally suited for elements with pitch closer to λ/2 in order to reduce the potential for interference due to grating lobes.

According to an alternate embodiment of the present disclosure, the ultrasound system reads the recorded performance data and determines whether or not a particular imaging mode is available for the connected probe 26. In some cases, the system may provide a message indicating that a particular probe 26 is not capable of providing suitable performance for the selected mode.

Applicants have described a method for ultrasound imaging comprising generating and emitting a test pattern of ultrasound transmission signals from a transducer probe; sensing the generated test pattern and identifying one or more defective transducer elements of the transducer probe; recording data related to the one or more defective transducer elements and associating the recorded data with the transducer probe; and acquiring and displaying or storing at least one ultrasound image using the transducer probe and using the associated recorded data for the transducer probe.

Associating the recorded data with the transducer probe can comprise recording the data related to the one or more defective transducer elements on a memory circuit within the transducer probe. Alternately, associating the recorded data with the transducer probe can comprise recording the data related to the one or more defective transducer elements on a networked server that is in signal communication with an ultrasound apparatus. The method can allow the transducer to query the networked online server in order to refresh or update its own test results. The method can further comprise displaying a message to an operator indicating the state of a transducer probe. The method can further comprise adapting transducer probe behavior according to the recorded data on one or more defective transducer elements. Adapting transducer probe behavior can comprise changing the phases applied to operating transducer elements to compensate for defective transducer elements. The compensation can alter the emitted ultrasound pattern from a rectangular profile to a trapezoidal profile. The test pattern can be downloaded from an online server. Associating the recorded data with the transducer probe can comprise storing the recorded data on an online server.

According to an alternate aspect, Applicants have described a method for ultrasound imaging comprising generating and emitting a test pattern of ultrasound transmission signals from a transducer probe; sensing the generated test pattern and identifying one or more defective transducer elements of the transducer probe; recording data related to the one or more defective transducer elements and associating the recorded data with the transducer probe; and providing a message indicating that the probe is unsuitable for one or more ultrasound imaging modes. The method can further comprise storing one or more alternate transducer excitation schemes for an operating mode according to the identification of one or more defective transducer elements.

According to another alternate aspect, an ultrasound transducer probe has a memory that identifies the probe and that stores probe performance data. The memory can further store one or more operating sequences for transducer element excitation based on the stored probe performance data.

The present invention can be a software program. Those skilled in the art will recognize that the equivalent of such software may also be constructed in hardware. Because image manipulation algorithms and systems are well known, the present description will be directed in particular to algorithms and systems forming part of, or cooperating more directly with, the method in accordance with the present invention. Other aspects of such algorithms and systems, and hardware and/or software for producing and otherwise processing the image signals involved therewith, not specifically shown or described herein may be selected from such systems, algorithms, components and elements known in the art.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

A computer program product may include one or more storage medium, for example; magnetic storage media such as magnetic disk (such as a floppy disk) or magnetic tape; optical storage media such as optical disk, optical tape, or machine readable bar code; solid-state electronic storage devices such as random access memory (RAM), or read-only memory (ROM); or any other physical device or media employed to store a computer program having instructions for controlling one or more computers to practice the method according to the present invention.

The invention has been described in detail and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of the invention is indicated by the appended claims, and all changes that come within the meaning and range of equivalents thereof are intended to be embraced therein.

What is claimed is:

1. A method for ultrasound imaging, comprising:
generating and emitting a test pattern of ultrasound transmission signals from a transducer probe;
in response to sensing the generated test pattern, identifying one or more defective transducer elements of the transducer probe;
recording data related to the one or more defective transducer elements and associating the recorded data with the transducer probe;
displaying or storing at least one ultrasound image using the transducer probe; and
using the associated recorded data for the transducer probe.

2. The method of claim 1 wherein associating the recorded data with the transducer probe comprises recording the data related to the one or more defective transducer elements on a memory circuit in the transducer probe.

3. The method of claim 1 wherein associating the recorded data with the transducer probe comprises recording the data related to the one or more defective transducer elements on a networked server that is in signal communication with an ultrasound apparatus.

4. The method of claim 1 further comprising displaying a message to an operator indicating the state of a transducer probe.

5. The method of claim 1 further comprising adapting transducer probe behavior according to the recorded data on one or more defective transducer elements.

6. The method of claim 5 wherein adapting transducer probe behavior comprises changing the phases applied to operating transducer elements to compensate for defective transducer elements.

7. The method of claim 6 wherein adapting transducer probe behavior includes altering the emitted ultrasound pattern from a rectangular profile to a trapezoidal profile.

8. The method of claim 1 further comprising downloading the test pattern from an online server.

9. The method of claim 1 wherein associating the recorded data with the transducer probe comprises storing the recorded data on an online server.

10. A method for ultrasound imaging comprising:
generating and emitting a test pattern of ultrasound transmission signals from a transducer probe;
sensing the generated test pattern and identifying one or more defective transducer elements of the transducer probe;
recording data related to the one or more defective transducer elements and associating the recorded data with the transducer probe; and
providing a message indicating that the probe is unsuitable for one or more ultrasound imaging modes.

11. The method of claim 10 further comprising storing one or more alternate transducer excitation schemes for an operating mode according to the identification of one or more defective transducer elements.

12. An ultrasound transducer probe having associated memory that identifies the probe and that stores probe performance data.

13. The transducer probe of claim 12 wherein the memory stores one or more operating sequences for transducer element excitation based on the stored probe performance data.

14. The transducer probe of claim 12 wherein the associated memory is stored on a networked server.
